(19) United States
(12) Patent Application Publication
(10) Pub. No.: US 2015/0025361 A1
(43) Pub. Date: Jan. 22, 2015

(54) Breast Coil with a Mechanical Height Adjustment

(71) Applicant: Stephan Biber, Erlangen (DE)

(72) Inventor: Stephan Biber, Erlangen (DE)

(21) Appl. No.: 14/334,057

(22) Filed: Jul. 17, 2014

(30) Foreign Application Priority Data
Jul. 18, 2013 (DE) 102013214130.0

(51) Int. Cl. G01R 33/34; A61B 5/055; A61B 5/00

(52) U.S. Cl. CPC G01R 33/34084; A61B 5/708; A61B 5/0555
USPC 600/415

(57) ABSTRACT

A local coil for a magnetic resonance imaging (MRI) system is a breast coil that includes at least one height adjustment apparatus.

### RELATED APPLICATIONS

This application claims the benefit of German Patent Application No. DE 102013214130.0, filed Jul. 18, 2013. The entire contents of the priority document are hereby incorporated herein by reference.

### TECHNICAL FIELD

The present teachings relate generally to local coils.

### BACKGROUND

Magnetic resonance imaging (MRI) devices for examining objects or patients using magnetic resonance imaging are described, for example, in DE 10314215B4.

### SUMMARY AND DESCRIPTION

The scope of the present invention is defined solely by the appended claims, and is not affected to any degree by the statements within this summary.

The present embodiments may obviate one or more of the drawbacks or limitations in the related art. For example, in some embodiments, a local coil for an MRI device may be optimized.

### BRIEF DESCRIPTION OF THE DRAWINGS

FIG. 1 shows a cross-sectional view of an example of a height adjustment apparatus for an MRI breast coil.

FIG. 2 shows a cross-sectional view of an example of a height adjustment apparatus for an MRI breast coil.

FIG. 3 shows a cross-sectional view in a sagittal plane of an example of a height adjustment apparatus for an MRI breast coil.

FIGS. 4-6 show examples of breast coils arranged at different heights in an MRI bore.

FIG. 7 shows a schematic illustration of an example of an MRI system.

### DETAILED DESCRIPTION

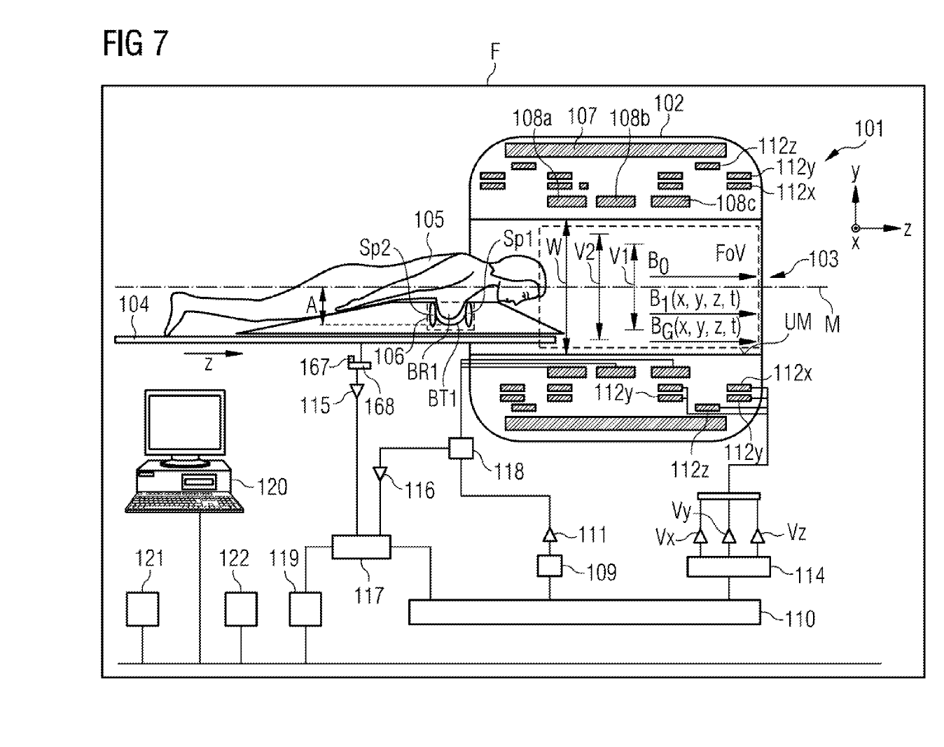

FIG. 7 shows a magnetic resonance imaging (MRI) device 101 in a shielded room or Faraday cage F. The device 101 includes a whole body coil 102 that, in some embodiments, includes a tubular space 103. A patient couch 104 with an examination object 105 (e.g. a patient), with or without local coil arrangement 106, may be driven in the direction of the arrow z to generate recordings of the patient 105 by an imaging method. In some embodiments, a local coil arrangement 106 is arranged on the patient. Recordings of a portion of the body 105 in a local region of the MRI (also referred to as field of view or FOV) may be generated by the local coil arrangement. Signals from the local coil arrangement 106 may be evaluated (e.g., converted into images, stored, or displayed) by an evaluation device (168, 115, 117, 119, 120, 121, etc.) of the MRI 101. The evaluation device may be connected to the local coil arrangement 106 by, for example, coaxial cables, a radio link 167, or the like.

In order to use a MRI device 101 to examine a body 105 (e.g., an examination object or a patient) by magnetic resonance imaging, different magnetic fields that are matched to one another in temporal and spatial characteristics are radiated onto the body 105. A strong magnet (e.g., a cryomagnet 107) in a measurement cabin with an opening 103 that, in some embodiments, is tunnel-shaped may generate a strong static main magnetic field B₀ (e.g., having a strength of 0.2 Tesla to 3 Tesla or greater). A body 105 to be examined is supported by a patient couch 104 and driven into a region of the main magnetic field BO that is substantially homogeneous in the observation region field of view (FOV). The nuclear spins of atomic nuclei of the body 105 are excited by magnetic radiofrequency excitation pulses B1 (x, y, z, t) that are radiated by a radiofrequency antenna (and/or, optionally, a local coil arrangement). The radiofrequency antenna is depicted in a greatly simplified manner as a multi-part body coil 108 (e.g., 108a, 108b, 108c). By way of example, radiofrequency excitation pulses are generated by a pulse generation unit 109 that is controlled by a pulse sequence control unit 110. After amplification by a radiofrequency amplifier 111, the radiofrequency excitation pulses are conducted to the radiofrequency antenna 108. The radiofrequency system is shown schematically in FIG. 7. In a magnetic resonance imaging device 101, more than one pulse generation unit 109, more than one radiofrequency amplifier 111, and more than one radiofrequency antennas 108a, 108b, 108c may be used.

The magnetic resonance imaging device 101 further includes gradient coils 112x, 112y, 112z. Magnetic gradient fields B_{G} (x, y, z, t) are radiated by the gradient coils during a measurement for selective slice excitation and for spatial encoding of the measurement signal. The gradient coils 112x, 112y, 112z are controlled by a gradient coil control unit 114 (and, optionally, via amplifiers Vx, Vy, Vz). The gradient coil control unit 114, like the pulse generation unit 109, is connected to the pulse sequence control unit 110.

Signals emitted by the excited nuclear spins (e.g., of the atomic nuclei in the examination object) are received by the body coil 108 and/or at least one local coil arrangement 106. The signals are amplified by associated radiofrequency preamplifiers 116 and further processed and digitized by a reception unit 117. The recorded measurement data are digitized and stored as complex numbers in a k-space matrix. An associated MRI image may be reconstructed from the k-space matrix filled with values by a multidimensional Fourier transform.

For a coil that may be operated in both transmission mode and reception mode (e.g., the body coil 108 or a local coil 106), the correct signal transmission is regulated by an upstream transmission/reception switch 118. An image-processing unit 119 generates an image from the measurement data that is displayed to a user by an operating console 120 and/or stored in a storage unit 121. A central computer unit 122 controls the individual installation components.

In MR imaging, images with a high signal-to-noise ratio (SNR) may be recorded using local coil arrangements (e.g., coils, local coils). Local coil arrangements are antenna systems that are attached in the direct vicinity on (anterior) or under (posterior), or at or in, the body 105. During an MR measurement, the excited nuclei induce a voltage in the individual antennae of the local coil. The voltage is then amplified using a low-noise preamplifier (e.g., LNA, preamp) and transmitted to the reception electronics. In order to improve the signal-to-noise ratio even for high-resolution images, high-field installations (e.g., 1.5 Tesla to 12 Tesla or greater) may be used. If more individual antennae are connected to an MR reception system than there are receivers available, a switching matrix (also referred to as RCCS) may be installed between the reception antennae and receivers. The matrix routes the currently active reception channels (e.g., the channels that currently lie in the field of view of the magnet) to the available receivers. As a result, more coil elements may be connected than there are receivers available because, in the case of a whole body cover, only coils that are situated in the FOV or in the homogeneity volume of the magnet are read.

By way of example, an antenna system that may include one antenna element or, as an array coil, several antenna elements (e.g., coil elements) may be referred to as a local coil arrangement 106. In some embodiments, these individual antenna elements may be embodied as loop antennae (loops), butterfly coils, flex coils, or saddle coils. In some embodiments, a local coil arrangement includes coil elements, a preamplifier, additional electronics (e.g., standing wave traps etc.), a housing, and supports. The local coil arrangement may also include a cable with a plug for connecting to the MRI installation. A receiver 168 attached to the installation side filters and digitizes a signal received from a local coil 106 (e.g., by radio link etc.) and transmits the data to a digital signal-processing device. The digital signal-processing device may derive an image or a spectrum from the data obtained by a measurement and makes the image or spectrum available to the user (e.g., for subsequent diagnosis by the user and/or for storing).

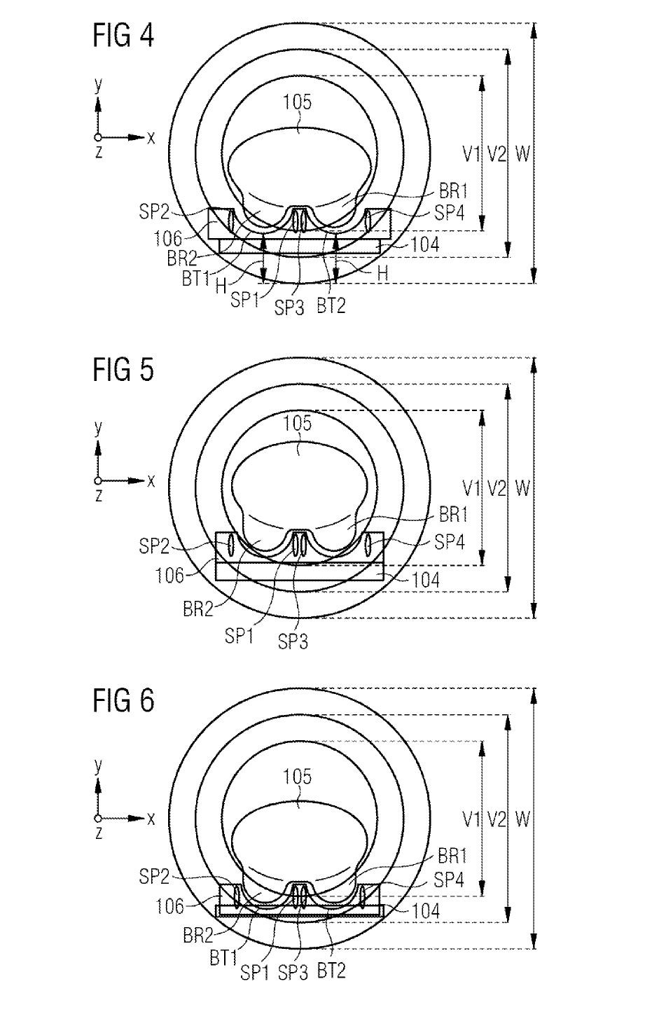

Each of FIGS. 4-6 depicts a partial cross-section in order to highlight spatial relationships. As shown in FIGS. 4-6, to increase the SNR and accelerate the measurement time, local coils 106 (e.g., arrays) with antennae Sp1-Sp4 are used by an MRI system 101 for examining different body regions of a body 105. The antennae Sp1-Sp4 may be active antenna structures with one or more antenna elements SP1, SP2, SP3, SP4 (also referred to as coils). The one or more antenna elements P1, SP2, SP3, SP4 are positioned (e.g., locally) in the vicinity of the body of the patient 105. Breast coils 106 may be used for examining a female breast BR1, BR2. During breast examination, a woman 105 is positioned on the breast coil 106 in a prone position. The breast coil 106 is arranged on a patient table 104 of the MRI device 101 and is conveyed thereon into a bore 103 (e.g., an opening) of the MR system 101. For horizontal field systems, bore openings W of 55-70 cm may be used. Within the bore 103, the examination volume FOV is restricted by the homogeneity of the main field magnet of the MRI device. There are two gradations: the volume V1 and the volume V2. The volume V1 has a homogeneity greater than 1 ppm (e.g., deviation from the nominal field). Good spectral fat saturation may be achieved within the zone V1. Within a larger volume V2 having 20-30 ppm deviation, MR imaging may, in principle, be performed but without spectral fat saturation techniques.

The height of the breast coil 106 may be set relative to the patient table 104, the upper edge of the bore entrance 103, and the first homogeneity volume V1 and/or the second homogeneity volume V2. Patients with relatively large body dimensions (e.g., with respect to torso diameter) in the y-direction may bump against the upper edge of the bore entrance 103, thereby reducing the quality of or preventing examination of the patient (e.g., in systems with only a 60-cm bore opening). Lowering the breast coil 106 downward is restricted mechanically by the patient table 104, and technically by the first homogeneity volume V1 and/or the second homogeneity volume V2 of the clear main field magnet internal diameter. If the anatomy BR1, BR2 of the patient to be examined slips downward out of the volume V1, there is a deterioration in the fat saturation. If the volume of the anatomy BR1, BR2 to be examined slips even further down, imaging in these regions may fail completely.

Breast coils 106 (e.g. in the case of a 60-cm MRI system) may be positioned in a region having a spine coil integrated into the patient table 104 in order to access, for example, an additional 3-7 cm of clear downward installation space. However, such a mechanically rigid solution may not address all contingencies. For example, if the breast coil 106 is seated high in the y-direction (e.g., such as the local coil 106 in FIG. 5 as compared to FIG. 4), the breast coil 106 may offer good image quality characteristics because both breast cups BT1, BT2 (e.g., recesses in the local coil 106 each of which is individually configured to receive one breast) of the breast coil 106 lie in the volume V1, as shown for example in FIG. 5. However, women with large (e.g., torso/chest area) dimensions may be examined poorly or not at all. By contrast, if the breast coil 106 is seated very low in the y-direction (e.g., such as the local coil 106 in FIG. 6 as compared to FIG. 4), a higher percentage of women may be examined although image quality problems in the case of spectral fat saturation may result. FIGS. 4-6 highlight examples of solutions for systems with different V1/V2 dimensions, with the inner circle V1 depicting a 1-ppm line and the outer circle V2 depicting a 30-ppm line. If the breast coil 106 were placed higher up in the y-direction in the system in FIG. 6 (e.g., as in FIG. 5), such as into a spinal coil recess in the patient couch 104, fewer patients would have adequate space due to the bore size but the breast would be within the fat-sat volume V. In such systems, a compromise may be made to the detriment of fat-sat image quality since parts of the breast coil cups BT1, BT2 protrude out of the volume V1 in FIG. 6.

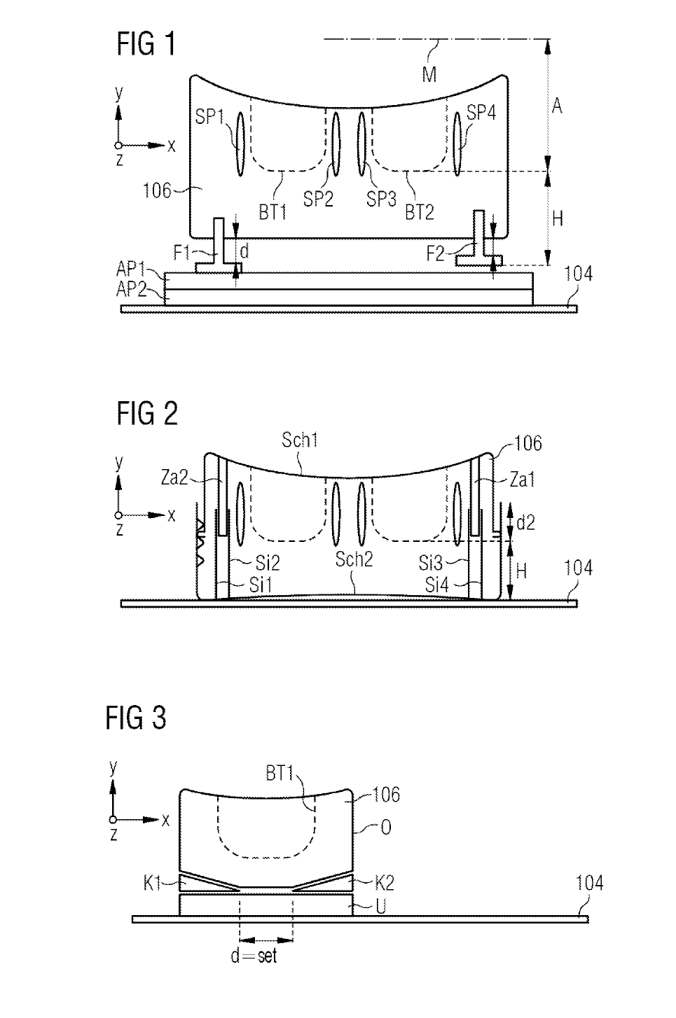

Each of FIGS. 1-3 shows an exemplary configuration of a height adjustment apparatus for a breast coil 106 in a magnetic resonance imaging device 101. The elements of the exemplary height adjustment apparatuses F1, F2; AP1, AP2; Za1, Za2; K1, K2 depicted in FIGS. 1-3 may be combined with one another in different ways to produce other embodiments that likewise fall within the scope of the present teachings.

A breast coil 106 may include at least one mechanical height adjustment apparatus F1, F2; AP1, AP2; Za1, Za2; K1, K2 configured to adjust the height H of the lowest regions of a first breast cup BT1 and a second breast cup BT2 of a breast coil 106 over the inner side of the central, lowermost inner wall cladding UM of the bore 103. Thus, the at least one mechanical height adjustment apparatus is configured to adjust the distance A of the lowermost regions of the first breast cup BT1 and the second breast cup BT2 of the breast coil 106 from the centerline M of the bore 103.

The breast coil 106 may be operated with high image quality for all patients 105 other than one of extraordinarily large body dimensions. This operation of the breast coil 106 with such high image quality is facilitated by the breast coil 106 being moved as far as possible in the direction of the isocenter M of the MRI device 101. Lower image quality may be acceptable for women with extraordinarily large body diameters who could otherwise not be examined at all, and may be achieved by moving the breast coil to a lower height H.

A height adjustment apparatus configured to adjust the height H of the first breast cup BT1 and the second breast cup BT2 of the breast coil 106 over a patient couch may be used to move parts of the breast coil or the whole breast coil 106 efficiently in the y-direction before and/or after positioning the patient 105.

As shown in FIG. 1, a height adjustment apparatus F1, F2; AP1, AP2; Za1, Za2; K1, K2 may include adapter plates—such as first adapter plate AP1 and second adapter plate AP2—between the couch 104 and the breast coil 106. Alternatively or in addition, the height adjustment apparatus may include adjustable wedges K1, K2 (e.g., as shown in FIG. 3), and/or toothed wheels, and/or Bowden cables, and/or extendable “feet” F1, F2 (e.g. as shown in FIG. 1), and/or pneumatic arrangements (e.g. for inflating or deflating an air bag), and/or pads that are compressed by weight (e.g. positioned at the feet or adapter plates), and/or other mechanical displacement apparatuses (e.g. as shown in FIG. 2) at the height H of the patient couch 104.

By way of example, as shown in FIG. 1, a first foot F1 and a second foot F2 may be provided instead of, or in addition to, the first adapter plate AP1 and the second adapter plate AP2 on the breast coil 106 as a height adjustment apparatus configured to adjust the height H of the first breast cup BT1 and the second breast cup BT2 of the breast coil 106 over a patient couch 104. The first foot F1 and the second foot F2 are vertically movable (e.g., in a y-direction) and may, for example, latch in at different heights in recesses and/or guide rails in the lower part of the breast coil 106 (e.g., using a tongue-and-groove or other mechanism).

As shown in FIG. 2, the upper part of the breast coil 106 includes a first guide pin Za1 and a second guide pin Za2 as a height adjustment apparatus for adjusting the height H of the lowermost points of the first breast cup BT1 and the second breast cup BT2 of the breast coil 106 over a patient couch 104. The first guide pin Za1 and the second guide pin Za2 are vertically movable (e.g., in a y-direction) and may, for example, latch into an elastic latching apparatus R at different heights in guide rails Si1, Si2, Si3, Si4 in the lower part of the breast coil 106 (e.g., using a tongue-and-groove mechanism). As shown in FIG. 2, the upper and lower part of the housing of the breast coil 106 may be embodied as a first tray Sch1 and a second tray Sch2 that lie on top of one another and are displaceable relative to one another (e.g., in the direction of the arrow d2 in FIG. 2).

As shown in FIG. 3, the height H of the lowermost points of the breast cup BT1 (and the breast cup BT2, not shown in FIG. 3) of the breast coil 106 over a patient couch 104 may, for example, be displaced by one or more wedges. For example, a first wedge K1 and a second wedge K2 may be moveable (e.g., in the direction of arrow d) under an upper part of the breast coil 106 and, in some embodiments, over a lower part U of the breast coil 106 connected (e.g., elastically or by at least one vertical rail, etc.) to the upper part O, thereby changing the height H of the breast coil over a patient couch.

Electromechanical drives configured to automatically set the height H may be used. In the case of electronically controllable or pneumatic systems, an optimum height H may be established in advance based on a weight of the patient 105 or by a sensor at the bore entrance or in the bore 103. The breast coil may initially be at the minimum height during insertion or at the isocenter M, and then brought to the optimum height with the aid of sensor information.

A height-adjustable breastplate may include a mechanical drive configured for height adjustment. In some embodiments, the drive is manually operable (e.g., by a lever and/or a screw that actuates a thread, thereby displacing the two trays of the breast coil toward one another).

A breast coil 106 in accordance with the present teachings may capitalize on magnetic homogeneity volume V1 by flexibly adapting the breast coil height H over the patient couch 104 to the bore opening and the patient size. As a result, a breast coil 106 may be provided with acceptable compromise between maximum patient size on one hand, and image quality in the case of fat saturation on the other.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding claim—whether independent or dependent—and that such new combinations are to be understood as forming a part of the present specification.

1. A local coil for a magnetic resonance imaging (MRI) system, wherein:
the local coil comprises a breast coil; and
the local coil comprises at least one height adjustment apparatus.

2. The local coil of claim 1, wherein the at least one height adjustment apparatus comprises height-adjustable feet.

3. The local coil of claim 1, wherein the at least one height adjustment apparatus comprises a plurality of adapters having identical or varying thicknesses, and wherein at least one adapter of the plurality of adapters is configured for assembly under the breast coil.

4. The local coil of claim 1, wherein the at least one height adjustment apparatus comprises a first tray and a second tray, wherein the first tray and the second tray are moveable with respect to one another and are configured for height adjustment.

5. The local coil of claim 1, wherein the at least one height adjustment apparatus comprises a latching apparatus configured for different heights.

6. The local coil of claim 1, wherein the at least one height adjustment apparatus comprises a plurality of wedges, and wherein each wedge of the plurality of wedges is configured for height adjustment.

7. The local coil of claim 1, wherein the at least one height adjustment apparatus comprises a pneumatic apparatus configured for height adjustment.

8. The local coil of claim 1, wherein the local coil is configured for height-adjustment by an electromechanical apparatus.

9. The local coil of claim 1, wherein the at least one height adjustment apparatus is configured to adjust a height of the local coil; wherein the at least one height adjustment apparatus is configured to adjust a height of one or more breast cups of the local coil relative to a patient couch, a bore, or the patient couch and the bore; or wherein the at least one height adjustment apparatus is configured to adjust the height of the local coil and the height of the one or more breast cups of the local coil relative to the patient couch, the bore, or the patient couch and the bore.

10. The local coil of claim 1, wherein the local coil comprises a breast coil comprising one or two breast cups, wherein each breast cup of the one or two breast cups is configured to receive a female breast.

11. The local coil of claim 2, wherein the at least one height adjustment apparatus comprises a plurality of adapters having identical or varying thicknesses, and wherein at least one adapter of the plurality of adapters is configured for assembly under the breast coil.

12. The local coil of claim 2, wherein the at least one height adjustment apparatus comprises a first tray and a second tray, wherein the first tray and the second tray are moveable with respect to one another and are configured for height adjustment.

13. The local coil of claim 3, wherein the at least one height adjustment apparatus comprises a first tray and a second tray, wherein the first tray and the second tray are moveable with respect to one another and are configured for height adjustment.

14. The local coil of claim 2, wherein the at least one height adjustment apparatus comprises a latching apparatus configured for different heights.

15. The local coil of claim 3, wherein the at least one height adjustment apparatus comprises a latching apparatus configured for different heights.

16. The local coil of claim 4, wherein the at least one height adjustment apparatus comprises a latching apparatus configured for different heights.

17. The local coil of claim 2, wherein the at least one height adjustment apparatus comprises a plurality of wedges, and wherein each wedge of the plurality of wedges is configured for height adjustment.

18. The local coil of claim 3, wherein the at least one height adjustment apparatus comprises a plurality of wedges, and wherein each wedge of the plurality of wedges is configured for height adjustment.

19. The local coil of claim 4, wherein the at least one height adjustment apparatus comprises a plurality of wedges, and wherein each wedge of the plurality of wedges is configured for height adjustment.

20. The local coil of claim 7 wherein the pneumatic apparatus comprises an inflatable air bag.
